(19) United States
(12) Patent Application Publication
(10) Pub. No.: US 2008/0031950 A1
(43) Pub. Date: Feb. 7, 2008

(54) Novel anelgesic combination

(75) Inventor: Ramesh Sesha, West Windsor, NJ (US)

Correspondence Address: Ramesh Sesha, 9113 Taylor Court, West Windsor, NJ, 08550, US

(73) Assignee: NECTID INC., PRINCETON, NJ (US)

(21) Appl. No.: 11/894,060

(22) Filed: Aug. 20, 2007

Related U.S. Application Data
(60) Provisional application No. 60/926,575, filed on Apr. 27, 2007.

(51) Int. Cl. A61K 9/24

(52) U.S. Cl. 424/472

(57) ABSTRACT

The invention discloses a method of administering a pharmaceutical combination comprising an NSAID and a slow release tramadol to a mammal in need of thereof. This invention further discloses an analgesic combination comprising an NSAID and a slow release tramadol for treating pain and pain related conditions.

### FIELD OF THE INVENTION

The present invention provides a method of treating pain and pain related conditions by administering to a patient in need thereof, a therapeutically effective amount of an NSAID and a slow release tramadol combination. The present invention further a pharmaceutical composition comprises of a slow release Tramadol and an immediate release NSAID such as Naproxen

### BACKGROUND OF THE INVENTION

Tramadol (FORMULA 1) is a centrally acting synthetic opioid analgesic. It is chemically (±) cis-2-[(dimethylamino) methyl]-1-(3-methoxyphenyl)cyclo-hexanol hydrochloride. It is commercially available in form of its hydrochloride salt (Formula II) as Ultram tablets. Tramadol is indicated in the treatment of the management of moderate to moderately severe pain in adults.

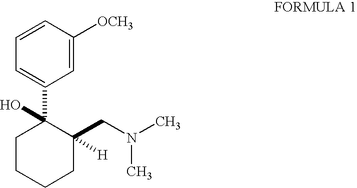

Tramadol is not an NSAID and doesn't have the increased risk of stomach ulceration and internal bleeding associated with non-steroidal anti inflammatory drugs (NSAID). However, it still has certain commonly reported side effects include nausea, constipation, dizziness, headache, drowsiness, and vomiting. Less commonly reported side effects include itching, sweating, dry mouth, diarrhea, rash, visual disturbances, and vertigo. It is desirable to prevent these side effects by prescribing lower doses of tramadol without compromising the extent of pain relief.

Most anti-inflammatory drugs have been associated with an increased risk of serious upper gastrointestinal complications. Overall, the risk is dose dependent and is greater with more than one anti-inflammatory drug taken simultaneously. Hence, whenever possible, anti-inflammatory drugs should be given in monotherapy. This is risk more pronounced in case on non-aspirin non-steroidal anti inflammatory (NA-NSAID) drugs Intake of NA-NSAIDs as a group has been consistently associated with a four- to five fold increase in UGIC¹. The risk is clearly dose dependent. The estimated pooled RRs in a recent meta-analysis were 3.0 (95% CI, 2.6-3.4) for low doses, 4.1 (95% CI, 3.6-4.5) for medium doses, and 6.9 (95% CI, 5.8-8.1) for high doses². There are research to indicate that NA-NSAID as a therapeutic class have a RR of 4.1 (95% CI, 3.6-4.8)¹

Naproxen (FORMULA 3) is a non-steroidal anti-inflammatory drug (NSAID) commonly used for the reduction of mild to moderate pain, fever, inflammation and stiffness caused by conditions such as osteoarthritis, rheumatoid arthritis, psoriatic arthritis, gout, ankylosing spondylitis, injury (like fractures), menstrual cramps, tendonitis, bursitis, and the treatment of primary dysmenorrhea.

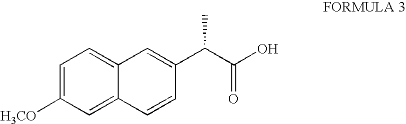

Naproxen and naproxen sodium, chemically known as (+)-(S)-2-(6-methoxynaphthalen-2-yl)propanoic acid, are marketed under various trade names including: Aleve, Anaprox, Naprogesic, Naprosyn, Naprelan, Synflex. Like other NSAIDs, naproxen is capable of producing disturbances in the gastrointestinal tract. Addition of a proton pump inhibitor such as esomeprazole will prevent this complication adverse effect. Naproxen in combination with a proton pump inhibitor is the safest NSAID combination.

As well documented in the literature, the NSAIDs such as naproxen can inhibit the excretion of sodium and lithium. Hence it is desirable to reduce their dosage to alleviate the patients of its side effects without comprising the extent of pain relief.

There is a strong unmet medical need for drugs that are free from side effects associated with both tramadol and NSAIDs. Considering these are drugs used over long term often by the elderly to manage pain that are often chronic, invention that help reduce the dosage of either or both of them without comprising the therapeutic benefits would fill these medical unmet needs.

Towards achieving this objective, this invention discloses a novel pharmaceutical combination comprising an NSAID and a slow release tramadol hydrochloride. The invention further discloses a method of treating pain and pain related disorder in a mammal comprising administering a combination comprising an NSAID and a slow release tramadol

U.S. Pat. No. 3,652,589 discloses a class of analgesic cycloalkanol-substituted phenol esters having a basic amine group in the cycloalkyl ring. The compound (1R,2R or 1S,2S)-2-[(dimethylamino) methyl]-1-(3-methoxyphenyl)-cyclohexanol, commonly known as tramadol, is specifically disclosed therein. A series of articles pertaining to the pharmacology, toxicology and clinical studies of tramadol are found in Arzneim, Forsch. (Drug Res.), 28(I), 114 (1978)³. Driessen et al., Arch. Pharmacol., 341, R104 (1990)⁴ disclose that tramadol produces its analgesic effect through a mechanism that is neither fully opioid-like nor non-opioid-like. The Abstracts of the VIth World Congress on Pain, Apr. 1-6 (1990)⁵, disclose that tramadol hydrochloride is an orally active pure agonist opioid analgesic. However, clinical experience indicates that tramadol lacks many of the typical side effects of opioid agonists, e.g., respiratory depression (W. Vogel et al., Arzneim Forsch. (Drug Res.), 28(I), 183 (1978))⁶, constipation (I. Arend et al., Arzneim. Forsch. (Drug Res.), 28(I), 199 (1978))⁷, tolerance (L. Flohe et, al., Arzneim. Forsch. (Drug Res.), 28(I), 213 (1978))⁸, and abuse liability (T. Yanagita, Arzneim. Forsch. (Drug Res.), 28(I), 158 (1978))⁹. When given at a dose of 50 mg by rapid i.v. injection, tramadol may produce certain side effects unique to tramadol including hot flushes and sweating. Despite these side effects, tramadol combination of non-opioid and opioid activity makes tramadol a very unique drug. Tramadol is currently being marketed by Grunenthal GMBH as an analgesic.

Opioids have for many years been used as analgesics to treat severe pain. They, however, produce undesirable side effects and as a result cannot always be given repeatedly or at high doses. The side effect problems are well documented in the literature. See, for example, J. Jaffe in “Goodman and Gilman's, The Pharmacological Basis of Therapeutics”, 8th edition; Gilman et al.; Pergamon Press, New York, 1990; Chapter 22; pages 522-573¹⁰ wherein it is disclosed that morphine and its congeners, e.g., codeine, hydrocodone and oxycodone, are opioid agonist analgesics that exhibit side effects such as respiratory depression, constipation, tolerance and abuse liability. As alternatives to using opioids, non-opioids such as aspirin and ibuprofen are used as analgesics. Ibuprofen, like aspirin, is not subject to the tolerance, addiction and toxicity of the opioid analgesics. However, ibuprofen, aspirin and other non-steroidal anti-inflammatory drugs (commonly referred to as NSAIDs) are only useful in relieving pain of moderate intensity, whereas the opioid analgesics are useful in relieving more intense pain; See Woodbury, D. and Fingl, E. in “The Pharmacological Basis of Therapeutics”, 5th Ed.; Goodman, L. and Gilman, A., Chapter 15, (1975)¹¹.

To reduce the side effect problems of opioids, opioids have been combined with other drugs including non-opioid analgesic agents, which lower the amount of opioid needed to produce an equivalent degree of analgesia. It has been claimed that some of these combination products also have the advantage of producing a synergistic analgesic effect. For example, A. Takemori, Annals New York Acad. Sci., 281,262 (1976)¹² discloses that compositions including combinations of opioid analgesics with drugs other than analgesics exhibit a variety of effects, i.e., sub additive (inhibitory), additive or super additive. R. Taber et al., J. Pharm. Expt. Thera., 169(1), 29 (1969)¹³ disclose that the combination of morphine and methadone, another opioid analgesic, exhibits an additive effect. U.S. Pat. No. 4,571,400 discloses that the combination of dihydrocodeine, an opioid analgesic, and ibuprofen, a non-opioid analgesic, provides super additive effects when the components are within certain ratios. See also U.S. Pat. Nos. 4,587,252 and 4,569,937, which disclose other ibuprofen opioid combinations. A. Pircio et al., Arch. Int. Pharmacodyn., 235, 116 (1978)¹⁴ report super additive analgesia with a 1:125 mixture of butorphanol, another opioid analgesic, and acetaminophen, a non-opioid analgesic, whereas a 1:10 mixture did not show any statistically significant super additive analgesia. Combinations of non-opioid analgesics have also been prepared to avoid the side effects associated with opioids, and the combinations are noted to have the benefit of requiring less of each ingredient and producing super additive effects. G. Stacher et. al., Int. J. Clin. Pharmacol. Biopharmacy, 17, 250 (1979)¹⁵ report that the combination of non-opioid analgesics, i.e., tolmetin (another NSAID) and acetaminophen, allows for a marked reduction in the amount of tolmetin required to produce analgesia. In addition, U.S. Pat. No. 4,260,629 discloses that an orally administered composition of acetaminophen and zomepirac, a non-opioid analgesic, in a particular weight ratio range produces a superadditive relief of pain in mammals. Furthermore, U.S. Pat. No. 4,132,788 discloses that 5-aroyl-1-(lower) alkylpyrrole-2-acetic acid derivatives, non-opioid analgesics, when combined with acetaminophen or aspirin exhibit super-additive ant arthritic activity. However, there have been warnings against the daily consumption of non-opioid analgesic mixtures and of the consumption of a single non-opioid analgesic in large amounts or over long periods (see, D. Woodbury and E. Fingl at page 349)¹¹. In addition, ibuprofen, aspirin and some other NSAIDs may cause gastrointestinal side effects especially if used repeatedly. See, for example, M. J. S. Langman, Am. J. Med. 84 (Suppl. 2A): 15-19, 1988)¹⁶; P. A. Insel in “The Pharmacological Basis of Therapeutics” 8th Ed.; Gilman, A. G. et al., Chapter. 26, pp. 664-668, 1990¹⁷.

US Patent Application 20050090517 discloses a combination COX 2 inhibitor or a pharmaceutically acceptable salt or derivative thereof and an opiate or a pharmaceutically acceptable salt or derivative thereof wherein COX 2 inhibitor is Meloxicam and opiate is Tramadol.

Tramadol is a centrally acting synthetic opioid analgesic. It is chemically (±) cis-2-[(dimethylamino)methyl]-1-(3-methoxyphenyl)cyclo-hexanol hydrochloride. It is commercially available in form of its hydrochloride salt (Formula I) as Ultram tablets.

Tramadol is indicated in the treatment of the management of moderate to moderately severe pain in adults.

Combinations of tramadol with acetaminophen and tramadol with Ibuprofen are the disclosures made in U.S. Pat. No. 5,336,691 and U.S. Pat. No. 5,516,803 respectively.

U.S. Pat. No. 6,284,274 assigned to Alza corporation discloses compositions comprising an opiate or non-opiate analgesic using an osmotic release technology.

U.S. Pat. No. 6,221,377 disclose a method of enhancing the analgesic, anti-inflammatory and anti-pyretic responses by administering the medicament with a pharmaceutically acceptable solution containing nitrous oxide. The presence of nitrous oxide enhances the efficacy of the medicament.

Tramadol is indicated in the treatment of the management of moderate to moderately severe pain in adults.

Combinations of tramadol with acetaminophen and tramadol with Ibuprofen are the disclosures made in U.S. Pat. No. 5,336,691 and U.S. Pat. No. 5,516,803 respectively.

U.S. Pat. No. 6,284,274 assigned to Alza corporation discloses compositions comprising an opiate or non-opiate analgesic using an osmotic release technology.

U.S. Pat. No. 6,221,377 disclose a method of enhancing the analgesic, anti-inflammatory and anti-pyretic responses by administering the medicament with a pharmaceutically acceptable solution containing nitrous oxide. The presence of nitrous oxide enhances the efficacy of the medicament.

U.S. Pat. No. 6,007,841 assigned to Algos discloses the potentiation of analgesic effectiveness of a narcotic agonist antagonist analgesic by co-administration of it with at least one non-toxic N-methyl D aspartate receptor antagonist. The N-methyl D aspartate receptor antagonist may comprise of drugs, which include tramadol, ibuprofen and acetaminophen. However an essential feature of the invention is the use of N-methyl D aspartate receptor antagonist to potentiate the effect of a narcotic agonist antagonist.

U.S. Pat. No. 5,945,416 and U.S. Pat. No. 5,998,434 both assigned to Lilly relate to combination compositions of olanzapine with analgesic drugs like ibuprofen, naproxen and others for treatment of pain. U.S. Pat. No. 6,254,887 discloses a controlled release formulation that achieves certain specific in vitro dissolution rates

U.S. Pat. No. 5,914,129 (Alexander et al) discloses magnesium containing analgesic compositions, which comprise a magnesium salt, a stimulant and an analgesic agent.

US 2003203028 assigned to Impax Pharmaceuticals is the subject of a multiplex drug delivery system comprising at least two immediate release components substantially enveloped by a second extended release component.

US Patent Application 20060099249 is the subject of a controlled release formulation comprising an opioid.

WO 0029022A1 assigned to Algos Pharmaceuticals claims a combination composition of a COX-2 inhibitor with a centrally acting narcotic analgesic or an agonist antagonist analgesic and tramadol.

WO 06053012 published and assigned to Azaya Therapeutics discloses a pharmaceutical composition comprising an analgesic combination comprising a) an NMDA antagonist or a pharmaceutically acceptable salt thereof, b) a methylxanthine or a pharmaceutically acceptable salt thereof and c) an opiate agonist, partial agonist or agonist/antagonist, or a pharmaceutically acceptable salt thereof.

WO 09850075 assigned to Algos Pharmaceuticals claims a combination composition of a COX-2 inhibitor with a N-methyl D aspartate receptor antagonist and/or a nontoxic substance that block at least one major intracellular consequence of NMDA receptor activation.

EP 08459989B1 assigned to Virginia Commonwealth University disclose a triple composition comprising an analgesic, a skeletal muscle relaxant/sedative and a N-methyl D aspartate receptor antagonist.

WO 05107467 assigned to Descartes Therapeutics claims a combination of an opioid, NSAID and a dopaminergic agent for treating pain or nociception.

The prior art, however, does not disclose a pharmaceutical composition comprising a slow release tramadol and an NSAID for treating a patient in need there of. Further prior art doesn't disclose a method of treating pain or pain related disorder comprising a method of administering to a mammal in need thereof, a pharmaceutical composition comprising a slow release tramadol and an NSAID

As there is a continuing need for analgesic medications that provide high efficacy pain relief with reduced undesirable effects. The present inventors while working on the analgesic combinations have surprisingly found the method of treating moderate to severe painful conditions associated with rheumatoid arthritis, osteoarthritis and the like, by administering to a subject in need thereof, a pharmaceutical analgesic combination comprising of 25-400 mg tramadol and 5-500 mg of an NSAID with pharmaceutically acceptable carrier so as to provide better pain management.

In one of the aspects of the present invention there is provided a method of treating moderate to severe pain by administering to a subject in need thereof, a pharmaceutical composition comprising 25-400 mg tramadol or a pharmaceutically acceptable salt or derivative thereof and 5-500 mg of an NSAID or a pharmaceutically acceptable salt or derivative thereof in admixture with pharmaceutically acceptable carrier. The pharmaceutical composition wherein tramadol and an NSAID are active ingredients that can be present in an immediate release form, extended release form or delayed release form along with pharmaceutically acceptable carrier.

This invention is advantageous as there will be a decreased dosing of the active ingredients to the patient coupled with the decrease in frequency of dosing over the individual drug and thus promote better patient compliance.

### BRIEF DESCRIPTION OF THE INVENTION

One object of the present invention is to provide methods, which can effectively be used in the treatment of pain and pain related diseases wherein the methods comprise administration of a therapeutically effective amount of an NSAID such as naproxen and administration of a therapeutically effective amount of a slow release Tramadol to a patient in need thereof.

The two analgesics i.e. an NSAID such as naproxen and a slow release tramadol may be co-administered or they may be administered separately as two medicaments.

Furthermore, the first drug may be administered in a regimen, which additionally comprises treatment with the second drug or third drug or the combination of first drug.

In yet another embodiment of the invention, a slow release tramadol and an NSAID such as naproxen are administered in suboptimal dosages.

In yet another embodiment of the invention t a slow release tramadol and an NSAID such as naproxen are administered in amounts and for a sufficient time to produce a synergistic effect.

### DETAILED DESCRIPTION OF THE INVENTION

The term “co-administration” as used herein means administration of the two compounds to the patient within a period of one month. The term includes separate administration of two medicaments each containing one of the compounds as well as simultaneous administration whether or not the two compounds are combined in one formulation or whether they are in two separate formulations.

The term “effective amount” as used herein means a dosage which is sufficient in order for the treatment of the patient to be effective compared with no treatment. The term “NSAID” as used in this specification means any non-steroidal anti-inflammatory drug including but not limited examples such as Celecoxib, Diclofenac, Diflunisal, Etodolac, Fenoprofen, Flurbirofen, Ibuprofen, Indomethacin, Ketoprofen, Ketorolac, Mefenamic Acid, Meloxicam, Nabumetone, Neproxen, Oxaprozin, Piroxicam, Sulindac and Tolmetin

The term “medicament” as used herein means a pharmaceutical composition suitable for administration of the pharmaceutically active compound to a patient.

The term “suboptimal dosage” us used herein means a dosage which is below the optimal dosage for that compound when used in single-compound therapy.

The term “additive effect” as used herein means the effect resulting from the sum of the effects obtained from the individual compounds.

The term “synergistic effect” as used herein means an effect which is greater than the additive effect which results from the sum of the effects of the two individual compounds.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “pain and pain related conditions” as used herein is defined as any pain due to a medical conditions including neuropathic pain, osteoarthritis, rheumatoid arthritis, fibromyalgia, and back, musculoskeletal pain, Ankylosing spondylitis, juvenile rheumatoid arthritis, migraines, dental pain, abdominal pains, ischemic pain, postoperative pain or because of an anesthetic or surgical contrition

The term “extended release material” as present in the inner solid particulate phase and the outer solid continuous phase refers to one or more hydrophilic polymers and/or one or more hydrophobic polymers and/or one or more other type hydrophobic materials, such as, for example, one or more waxes, fatty alcohols and/or fatty acid esters. The “extended release material” present in the inner solid particulate phase may be the same as or different from the “extended release material” present in the outer solid continuous phase.

The term “slow-release” here applies to any release from of a formulation that is other than an immediate release wherein the release of the active ingredient is slow in nature. This includes various terms used interchangeably in the pharmaceutical context like extended release, delayed release, sustained release, controlled release, timed release, specific release, targeted release etc

The term “candidate for sustained release” encompasses all the characteristics of a drug which make it a candidate for formulating it into an extended release fashion like a short elimination half life and consequent dosing of more than once a day, a single dose product given in an extended fashion to achieve better clinical results and avoid side effects associated with an immediate release etc

The term “binding agent” as used in this specification, refers to any conventionally known pharmaceutically acceptable binder such as polyvinyl pyrrolidone, hydroxypropyl cellulose, hydroxyethyl cellulose, hydroxypropyl methylcellulose, ethylcellulose, polymethacrylate, polyvinylalcohol, waxes and the like. Mixtures of the aforementioned binding agents may also be used. The preferred binding agents are water soluble materials such as polyvinyl pyrrolidone having a weight average molecular weight of 25,000 to 3,000,000. The binding agent may comprise approximately about 0 to about 40% of the total weight of the core and preferably about 3% to about 15% of the total weight of the core. In one embodiment, the use of a binding agent in the core is optional.

The term “pharmaceutically acceptable derivative” means various pharmaceutical equivalent isomers, enantiomers, complexes, salts, hydrates, polymorphs, esters etc of duloxetine

The term “therapeutically effective amount” means an amount that elicits a biological response in a mammal including the suboptimal amount

The term “hydrophilic polymers” as used in this specification include, but are not limited, to hydroxypropylmethylcellulose, hydroxypropylcellulose, sodium carboxymethylcellulose, carboxymethylcellulose calcium, ammonium alginate, sodium alginate, potassium alginate, calcium alginate, propylene glycol alginate, alginic acid, polyvinyl alcohol, povidone, carbomer, potassium pectate, potassium pectinate, etc

The term “hydrophobic polymers” as used in this specification include, but are not limited, to ethyl cellulose, hydroxyethylcellulose, ammonio methacrylate copolymer (Eudragit RL™ or Eudragit RS™), methacrylic acid copolymers (Eudragit L™ or Eudragit S™), methacrylic acid-acrylic acid ethyl ester copolymer (Eudragit L 100-5™), methacrylic acid esters neutral copolymer (Eudragit NE 30D™), dimethylaminoethylmethacrylate-methacrylic acid esters copolymer (Eudragit E 100™), vinyl methyl ether/malefic anhydride copolymers, their salts and esters (Gantrez™) etc.

Other hydrophobic materials which may be employed in the inner solid particulate phase and/or outer solid continuous phase include, but are not limited, to waxes such as beeswax, carnauba wax, microcrystalline wax, and ozokerite; fatty alcohols such as cetostearyl alcohol, stearyl alcohol; cetyl alcohol myristyl alcohol etc; and fatty acid esters such as glyceryl monostearate, glycerol monooleate, acetylated monoglycerides, tristearin, tripalmitin, cetyl esters wax, glyceryl palmitostearate, glyceryl behenate, hydrogenated castor oil, etc.

The present invention discloses a combination comprising an NSAID and a slow release tramadol. According to the invention the combination preferably contains a therapeutically effective amount of tramadol or a pharmaceutically acceptable salt thereof, wherein the tramadol is suitably in the range of from 50 to 800 mg, especially 100, 200, 300, 400 to 600 mg (calculated as tramadol hydrochloride) per dosage unit and therapeutically effective amount of an NSAID in the range from about 2 mg to about 500 mg.

The tramadol material is any one of (1R,2R or 1S,2S)-(dimethylaminomethyl)-1-(3-methoxyphenyl)-cyclohexanol (tramadol), its N-oxide derivative (“tramadol N-oxide”), and its O-desmethyl derivative (“O-desmethyl tramadol”) or mixtures thereof. It also includes the individual stereoisomers, mixtures of stereoisomers, including the racemates, pharmaceutically acceptable salts of the amines, such as the hydrochloride salt, solvates and polymorphs of the tramadol material. Tramadol is commercially available from Grunenthal or may be made by the process described in U.S. Pat. No. 3,652,589, which is herein incorporated by reference.

The combination according to the invention may be presented, for example, as granules, spheroids, pellets, multiparticulates, capsules, tablets, sachets, controlled release suspensions, or in any other suitable dosage form incorporating such granules, spheroids, pellets or multiparticulates.

The one or more of active ingredient in the combination according to the present invention may suitably be incorporated in a matrix. This may be any matrix, known to a person skilled the art, that affords slow release tramadol over at least a twelve hour period and preferably that affords in-vitro dissolution rates and in vivo absorption rates of tramadol within the therapeutically effective ranges. The combination according to the present invention may preferably use a slow release matrix. Alternatively, normal release matrices having a coating which provides for slow release of the tramadol may be used.

The slow release matrix employed in the combination of this invention may also contain other pharmaceutically acceptable ingredients which are conventional in the pharmaceutical art such as diluents, lubricants, binders, granulating aids, colorants, flavourants, surfactants, pH adjusters, anti-adherents and glidants, e.g. dibutyl sebacate, ammonium hydroxide, oleic acid and colloidal silica. Any known diluent e.g. microcrystalline cellulose, lactose and dicalcium phosphate may be used to prepare this combination. Suitable lubricants are e.g. magnesium stearate and sodium stearyl fumarate. Suitable binding agents are e.g. hydroxypropyl methyl cellulose, polyvidone and methyl cellulose. Suitable disintegrating agents are starch, sodium starch glycolate, crospovidone and croscarmellose sodium.

The surface actives that are suitable for this invention are Poloxamer 188®, polysorbate 80 and sodium lauryl sulfate. The suitable flow aids for this invention are talc colloidal anhydrous silica. Similarly, the suitable water soluble polymers that may be used to prepare the matrix are PEG with molecular weights in the range 1000 to 6000. The combination comprising the slow release tramadol according to the invention may conveniently be film coated using any film coating material conventional in the pharmaceutical art but preferably an aqueous film coating is used.

Alternatively, the combination comprising a slow release tramadol and an NSAID as per this invention may comprise a normal release matrix having a slow release coating. Preferably the combination comprises film coated spheroids containing the active ingredient and a spheronising agent. The spheronising agent may be any suitable pharmaceutically acceptable material which may be spheronised together with the active ingredient to form spheroids. A preferred spheronising agent as per this invention is microcrystalline cellulose. The microcrystalline cellulose used may suitably be, for example, Avicel PH 101 or Avicel PH 102 (Trade Marks, FMC Corporation). The spheroids may optionally contain other pharmaceutically acceptable ingredients conventional in the pharmaceutical art such as binders, bulking agents and colorants. Suitable binders may include water soluble polymers, water soluble hydroxyalkyl celluloses such as hydroxypropylcellulose or water insoluble polymers (which may also contribute controlled release properties) such as acrylic polymers or copolymers for example ethylcellulose. Suitable bulking agents include lactose.

The spheroids are coated with a material which permits release of the active ingredient at a slow rate in an aqueous medium. Suitable slow release coating materials that may be used in this invention include water insoluble waxes and polymers such as polymethylacrylates (for example Eudragit polymers, Trade Mark) or water insoluble celluloses, particularly ethylcellulose. Optionally, water soluble polymers such as polyvinylpyrrolidone or water soluble celluloses such as hydroxypropylmethylcellulose or hydroxypropylcellulose may be included. Optionally other water soluble agents such as polysorbate 80 may be added.

The following Examples 1 and 2 are shown for illustrating the invention related to combination comprising a slow release tramadol and an NSAID. According to this invention where we have used a specific NSAID Naproxen only as an example for illustrative purposes and these examples in no way limit the scope of the invention. The person skilled in the art will know how the combination may be modified using other NSAIDs such as Celecoxib, Diclofenac, Diflunisal, Etodolac, Fenoprofen, Flurbirofen, Ibuprofen, Indomethacin, Ketoprofen, Ketorolac, Mefenamic Acid, Meloxicam, Nabumetone, Naproxen, Oxaprozin, Piroxicam, Sulindac and Tolmetin and using other manufacturing methods known in the art. In general, the invention disclosed in this patent can be manufactured using the range listed in Table 1

TABLE 1
Slow Release Tramadol Hydrochloride and Naproxen

| | Quantity |
|---|---|
| First Active core | |
| Tramadol Hydrochloride | 50-250 MG |
| Microcrystalline Cellulose | 10-25% |
| Colloidal Silicon Doxide | 0.5-2.0% |
| Polyvinylpyrrolidone or PVA | 2-5% |
| Hydrogenated Vegitable Oil | 2-10% |
| Water | Q.S |
| Coat | |
| Ethyl Cellulose Aqueous Dispersion | 20-70% |
| Polyvinylpyrrolidone or HPMC | 20-40 |
| PEG 400 | 10-30% |
| Water | |
| Second Active Ingredient Layer | |
| Neproxen | 50-500 MG |
| Povidone K 30 USP | 2-10% |
| Microcrystalline cellulose | 2-10% |
| Croscaramellose sodium | 2-10% |
| Magnesium Stearate | 0.5-2% |

The Table 2 illustrates an example (Example 1) we used to manufacture a combination comprising a slow release tramadol hydrochloride core and an immediate release naproxen layer

### EXAMPLE 1

TABLE 2
Combination of slow release tramadol
100 mg and naproxen 250 mg tablets

| | Example 1 |
|---|---|
| First Active Ingredient mg/tablet | |
| Tramadol Hydrochloride | 100.0 |
| Microcrystalline Cellulose | 10.0 |
| Colloidal Silicon Dioxide | 1.5 |
| Polyvinylpyrrolidone | 4.5 |
| Hydrogenated Vegetable Oil | 5.0 |
| Water | Q.S |
| Coat | |
| Ethylcellulose Aqueous Dispersion | 50.00 |
| Opaspray YS -1 7006 | 5.00 |
| Water* | Q.S |
| Second Active Ingredient | |
| Naproxen | 250.0 |
| Povidone K 30 USP | 12 mg |
| Microcrystalline cellulose | 25 mg |
| Crosscaramellose sodium | 15 mg |
| Magnesium Stearate | 3 mg |
| Water* | q.s. |

*Removed during processing

Table 3 illustrates an example (Example 2) of a combination comprising a tramadol hydrochloride core and an immediate release naproxen at lower doses

TABLE 3
Combination of slow release tramadol
80 mg and naproxen 175 mg tablets

| | Example 1 |
|---|---|
| First Active Ingredient mg/tablet | |
| Tramadol Hydrochoride | 80.0 |
| Microcrystalline Cellulose | 8.0 |
| Colliodal Silicon Dioxide | 1.2 |
| Polyvinylpyrrolidone | 43.7 |
| Hydrogenated Vegetable Oil | 4.0 |
| Water | Q.S |
| Coat | |
| Ethylcellulose Aqueous Dispersion | 40.00 |
| Opaspray YS -1 7006 | 4.00 |
| Water* | Q.S |
| Second Active Ingredient | |
| Naproxen | 175.0 |
| Povidone K 30 USP | 12 mg |
| Microcrystalline cellulose | 17 |
| Crosscarmellose sodium | 11 |
| Magnesium Stearate | 2.2 |
| Water* | Q.S |

*Removed during processing

These examples are illustrative and are not intended to define the scope of the invention. A person skilled in the art would easily modify the invention by adding, substituting or removing specific ingredients.

Manufacturing Process of Slow Release Tramadol Hydrochloride and Naproxen

The combination comprising a slow release tramadol hydrochloride tablets and naproxen were manufactured in two phases using standard granulation and coating processes. In phase I, the Tramadol Hydrochloride was formulated into a core which was further coated with slow release coat to get a slow release tramadol core. In Phase II, the above prepared coated slow release Tramadol hydrochloride core was coated with an immediate release layer comprising Naproxen. The details are given below;

Phase I;

Core preparation: Tramadol HCl is mixed with microcrystalline cellulose and colloidal silicone dioxide and one or mixture of filler and granulated using suitable method known in the art using a binder solution comprising polyvinylpyrrolidone or polyvinyl alcohol. The granulated tramadol hydrochloride was dried and screened. This is further lubricated using hydrogenated vegetable oil with or without glidant. The lubricated blend is compressed into tablets using a compression machine.

Coating Solution and Coating: The coating solution is prepared using aqueous dispersion of water insoluble water permeable polymer of Ethylcellulose with water soluble polymer of polyvinylpyrrolidone or hydroxy propyl methyl cellulose. Polyethylene glycol mixture prepared using propeller stirrer and the same is homogenized using suitable homogenizer. The core tablets are coated using coating solution using standard coater like O'Hara pan coater tip set at 4″ at a spray rate of 25 mL/gun/min, exhaust temperature of around 45° C., an atomization pressure from 10-35 psi at a pan speed of 5-8 rpm, using airflow 350 CFM.

Phase II:

In phase II, Naproxen formulation prepared using granulation technique known in the art and then blended with disintegrant and lubricant.

Final Formulation Tramadol slow release tablets prepared in Phase I is coated with lubricated blend of Naproxen formulation using compression coating machine where Tramadol slow release tablets is used as a core and an immediate layer of Naproxen formulation forms an outer layer.

The naproxen coating was applied to coated 100 mg tramadol hydrochloride tablets using the above mentioned coater. Over this neproxen coated seal coated 100 mg tramadol hydrochloride tablets, color coating was done using similar coat. The spraying was done at a temperature of 46-47° C., atomization pressure of 40-60 psi at a spray rate of 180 grams per minute/three guns. The pan speed was at 4-8 rpm and air volume of 1000±100.

Finally, optionally color coated tablets were dried and polished using Cindrella wax and the finished final tablets were packaged in a HDPE bottle with a suitable desiccant and subjected appropriate stability and clinical studies. In vitro dissolution studies were conducted herein for determining the in vitro dissolution profile of Tramadol Hydrochloride in the combination as per conditions listed in Table 6. In Example 1, we used a combination comprising 100 mg of slow release tramadol and 250 mg naproxen. In Example 2, we prepared a combination comprising 80 Mg of slow release tramadol and 175 mg of naproxen.

### Method of Administration

The present inventions disclosed in this specification further include a method of treating pain and pain related conditions. This was established using a well controlled human clinical trial. The method of treating pain and pain related conditions by using a combination disclosed by this invention has been established in a long-term controlled clinical evaluation. A typical study determined the efficacy of tramadol and meloxicam as monotherapy and a combination of naproxen and a slow release tramadol for the treatment of pain and pain related conditions

### Clinical Trials

### 1. Drugs

Tramadol hydrochloride: Generic Tramadol Hydrochloride tablets 50 mg,
Naproxen: Generic Neproxen tablets 250 mg
Slow Release Tramadol Hydrochloride: a) Example 1, b) Ultram ER 100 mg

### 2. Treatment Combination

Treatment Drugs per day per patient
1. Treatment A; Tramadol hydrochloride 100 mg
2. Treatment B: Naproxen 250 mg
3. Treatment C: Example 1
4. Treatment D: Ultram ER 100 mg+Naproxen 250 mg
5. Treatment E: Example 2

### 3. Dosage

The administered dosage comprised either tramadol hydrochloride (100 mg) or naproxen 250 mg or a combination of naproxen (250 mg) and slow release tramadol hydrochloride (100 mg) selected from Treatments D or a combination of naproxen 250 mg and a slow release tramadol 100 mg (Treatment C) was administered once a day to the patients in a long-term clinical trial.

### 4. Patients

40 patients were used in a clinical trial were suffering from Chronic non-cancer pain (CNCP), defined as pain for longer than 6 months, including neuropathic pain, osteoarthritis, rheumatoid arthritis, fibromyalgia, and back and musculoskeletal pain were included in the clinical trial. No effort was made to segregate the patients based on the type of pain nor did we evaluate the extent of pain relief on the basis of pain type.

Those patients with migraines, dental pain, abdominal pains (from chronic pancreatitis, kidney stones, etc.) and ischemic pain from vascular disease were excluded because they are usually not classified as CNCP. Patients with history of addiction (alcohol or drugs) were excluded from the trials

### 5. Randomization

Randomization was performed with computer-generated random numbers in blocks of 10. Randomization codes of the monotherapy or combination therapy treatments were placed in sequentially numbered, opaque, sealed envelopes in the biopsy center. When a patient was recruited and consented, the next numbered envelope was opened by the operator, who had no knowledge of the randomization code before the treatment

### 6. Side Effects

There were four side effects that occurred significantly more often among those taking opioids than those in the placebo groups: constipation (RD 13%, 95% CI 9%-21%); nausea (RD 15% (13%-21%); dizziness or vertigo (RD 8% (5%-9%); somnolence or drowsiness (RD 9% (4%-11%); Risk differences for the other side effects noted (diarrhea, appetite loss, abdominal pain, dry mouth, headache, fatigue, blurred vision or accommodation disturbance, sleeplessness or insomnia, confusion, and sweating) were all statistically non-significant. There was no difference in the extent of side effects between the combination therapy and the monotherapy

Compared with naproxen used in the clinical trial, only 3 side effects occurred significantly more frequently with opioids: the RD for nausea was 11% (95% CI 4%-22%); constipation, 7% (1%-14%); and somnolence or drowsiness, 7% (0-10%). Risk differences for the other 12 side effects (vomiting, dizziness, dry skin, loss of appetite, abdominal pain, dry mouth, headache, fatigue, vision disturbance, insomnia, confusion and sweating) were not statistically significant.

### 7. Scale and Measurement

Pain was assessed using a visual analogue scale (VAS) graded from 0-100 mm. Pain scoring was performed every three hours after drug administration for a total of 48 hours.

The patients were taught with a standard method by the physician how to use a VAS scoring from 0 to 100 mm to grade the intensity of pain experienced during the treatment. On that scale, the left endpoint, 0, was defined as no pain and the right endpoint 100, as the worst pain the patient could imagine. There were no further marks on the line. The intensity of pain was indicated by the distance in millimeters from the left end. Patients were asked to grade the pain intensity immediately after the administration of drugs and every three hour after for 24 hours by drawing a vertical line over each VAS line. The doctors who prescribed the medicines obtained all the VAS forms for that specific patient.

### 8. Statistical Analysis

Data are expressed as means±SD. Differences in VAS were analyzed with use of the unpaired Student t test and differences in pain score reductions between the two groups were analyzed with use of the Mann-Whitney U test for the median difference. Similar statistical tests were used for between group comparisons of other outcome variables as appropriate. A two tailed P value of less than. 0.5 was considered to demonstrate statistical significance. SAS software was used for the statistical analysis.

We considered that a clinically significant benefit of using meloxicam and slow release tramadol would be a reduction in the pain score (VAS) of at least 15% compared to the monotherapy with either tramadol hydrochloride or naproxen or placebo. Alternatively, the dose reduction of at least 15% of either NSAID or tramadol when used as combination over the monotherapy as significant benefit

### 9. Results

The objectives of the inventions are met by the following results from the clinical trials.

Table 4 shows the patient's profile

Table 5 shows the VAS pain scores for Neproxen, tramadol, a fixed dose combination comprising naproxen and a slow release tramadol and for a co-administered combination of naproxen and Ultram ER

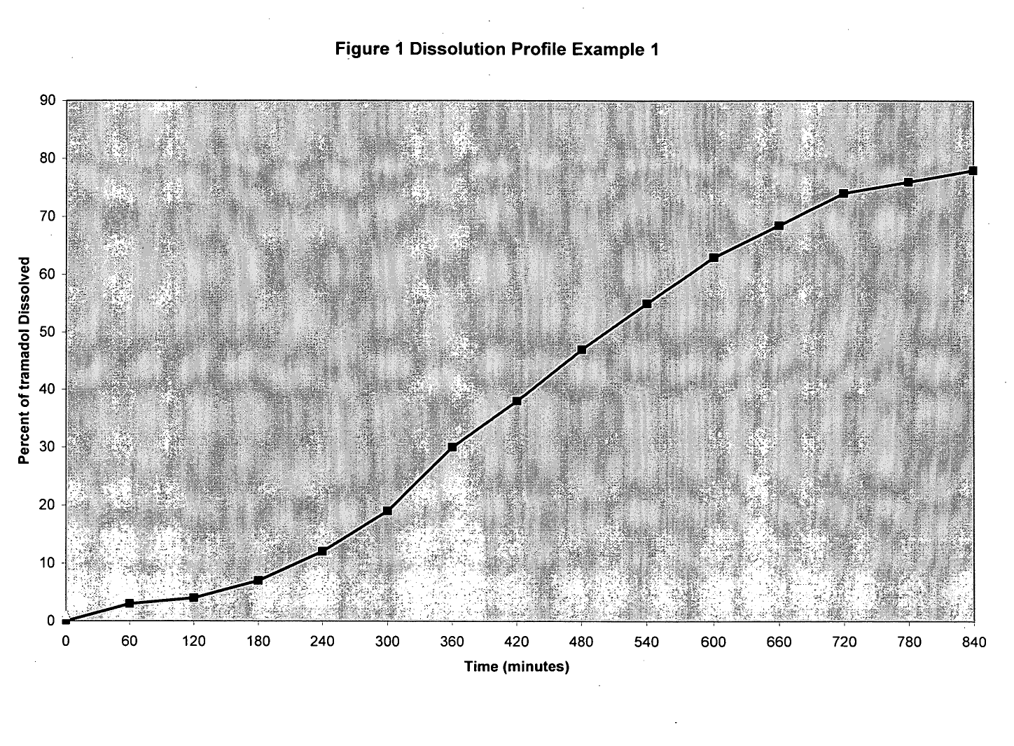

FIG. 1: Illustrates the in vitro dissolution profiles of Tramadol in Tablets formulated according to Example 1 in comparison with commercially available Reference

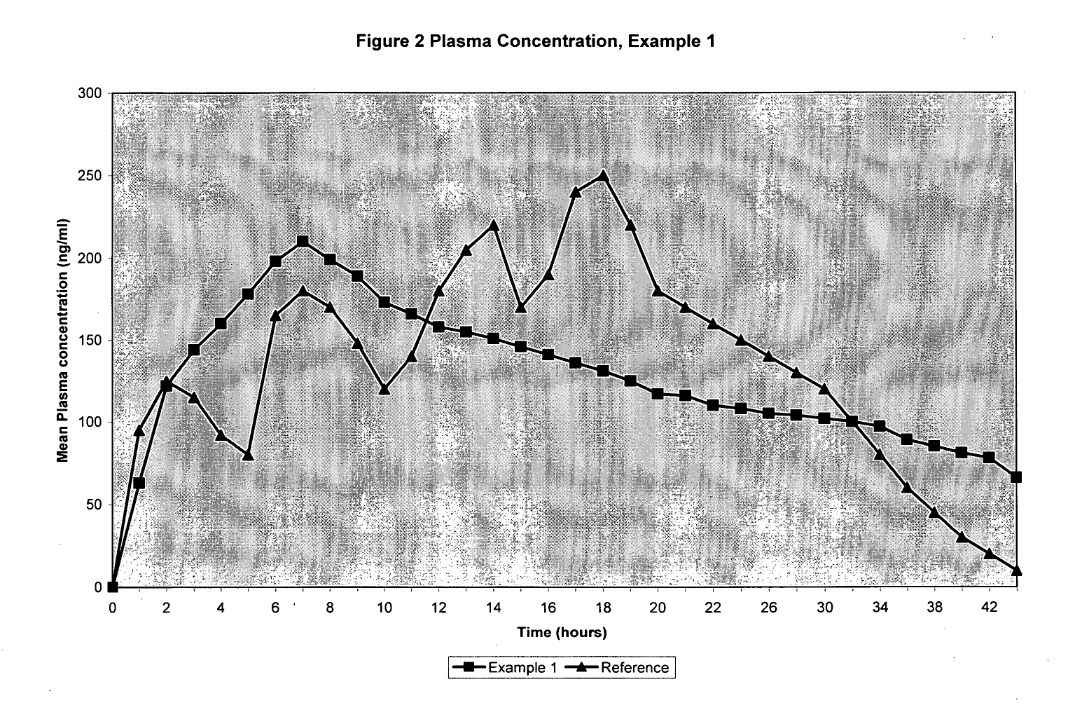

FIG. 2: Illustrates the mean plasma Tramadol concentrations (ng/ml) over time following administration of once a day Tablet (two times) formulated according to Example 1 in comparison with commercially available Reference Utrm 50 mg qid

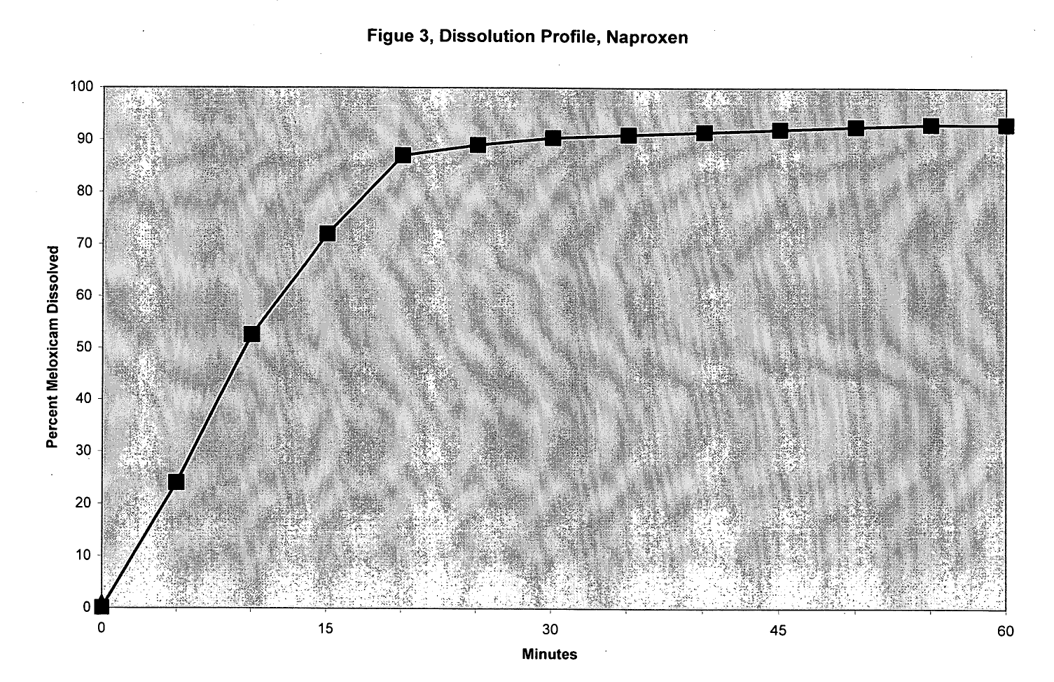

FIG. 3: Illustrates the in vitro dissolution profiles of Naproxen in tablets formulated according to Example 1

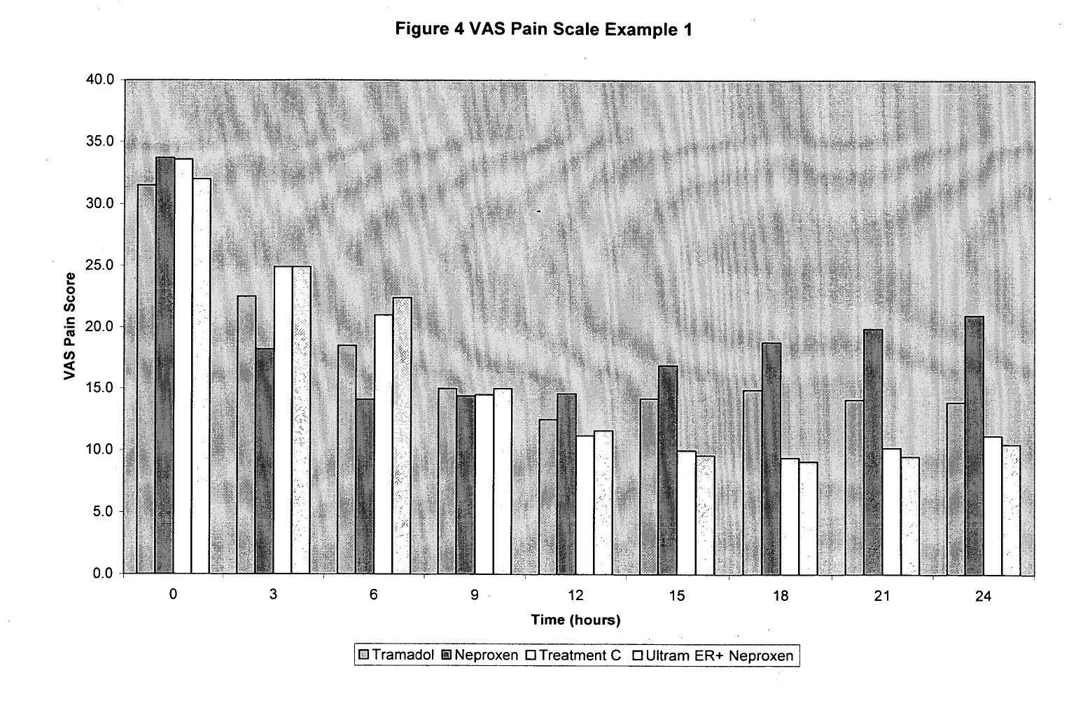

FIG. 4: VAS pain score for each of the treatment measured at an interval of every three hours over a period of 24 hours for naproxen, tramadol hydrochloride, a fixed dose combination comprising naproxen and a slow release tramadol (Example 1) and for a co-administered combination of naproxen and Ultram ER

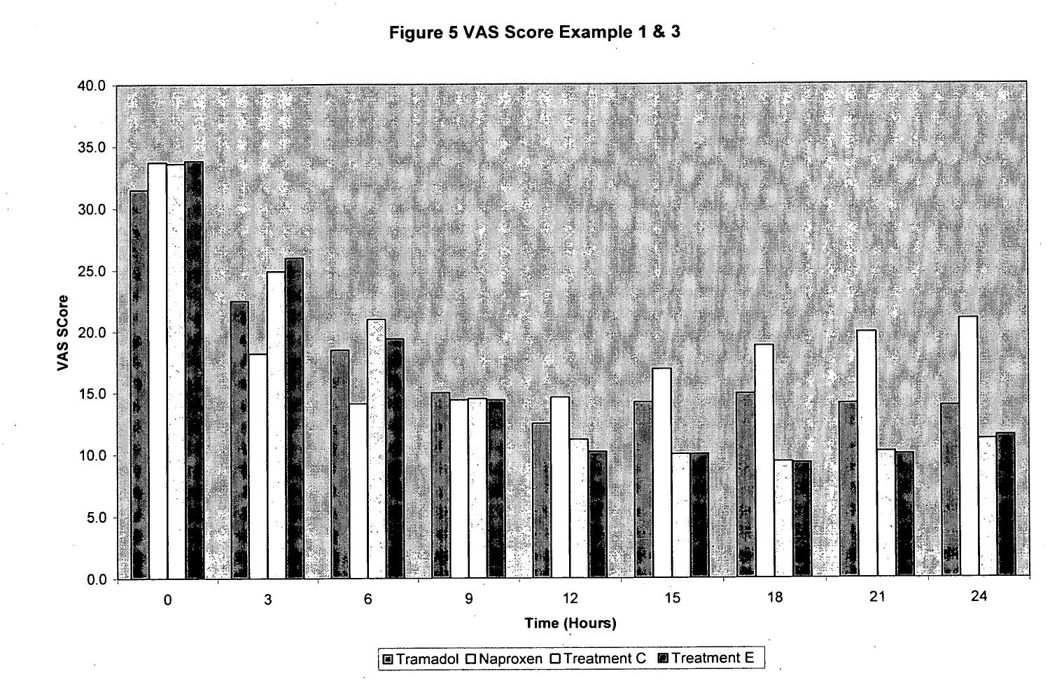

FIG. 5: VAS pain score comparison for tramadol and naproxen monotherapy with a combination comprising tramadol hydrochloride and naproxen at two different dosages (Example 1 and Example 2). This result demonstrates that it is possible to reduce the dosage of both tramadol and naproxen according to this invention without compromising the extent of pain relief

### 10. CONCLUSIONS

In summary, this invention is related to the novel pharmaceutical composition comprising a slow release tramadol hydrochloride and an immediate release NSAID. The invention further provides a method of treating pain and pain related diseases. The invention provides an analgesic combination comprising an NSAID and a slow release tramadol hydrochloride. For example: The invention was established by preparing a fixed dose combination of a slow release tramadol and immediate release naproxen. The efficacy was established using a long term human clinical trials with over 50 patients using a slow release tramadol and naproxen combination therapy. The combination therapy of naproxen and a slow release tramadol appears to be safe and well-tolerated and can result in significant therapeutic benefits. This combination is exemplified using a combination comprising naproxen (250 mg) and a slow release tramadol (100 mg) that was either co-administered or as a fixed dose (Example 1) twice a day to the patients in a long-term clinical trial. Still further, this invention demonstrates very surprisingly that a combination comprising a slow release tramadol and an immediate release naproxen is equally effective at lower doses than the doses administered individually.

The foregoing study establishes that the combination of an NSAID such as naproxen and a slow release tramadol results in a clinically significant and unexpected lowering of pain compared to either agent used alone. Further the study demonstrates that the present invention where the slow release tramadol reduces the dose of tramadol by 20% and that of naproxen by at least 33% without compromising the degree of pain relief to the patient

While certain preferred and alternative embodiments of the invention have been set forth for purposes of disclosing the invention, modifications to the disclosed embodiments may occur to those who are skilled in the art. Accordingly, the invention is intended to cover pharmaceutical compositions comprising a slow release tramadol and with all NSAIDs, all embodiments of the invention and modifications thereof which do not depart from the spirit and scope of the invention. Other NSAIDs like Celecoxib, Diclofenac, Diflunisal, Etodolac, Fenoprofen, Flurbirofen, Ibuprofen, Indomethacin, Ketoprofen, Ketorolac, Mefenamic Acid, Meloxicam, Nabumetone, Naproxen, Oxaprozin, Piroxicam, Sulindac and Tolmetin

### REFERENCES

1. The risk of upper gastrointestinal complications associated with nonsteroidal anti-inflammatory drugs, glucocorticoids, acetaminophen, and combinations of these agents, Luis Alberto García Rodríguezl and Sonia Hernández-Díaz, Arthritis Res. 2001; 3(2): 98-101
2. Hernández-Díaz S, García-Rodríguez L A. Overview of epidemiological studies published in the nineties on the association between non-steroidal anti-inflammatory drugs and upper gastrointestinal bleeding/perforation. Arch Internal Med. 2000; 160:2093-2099
3. Arzneim, Forsch. (Drug Res.), 28(I), 114 (1978).
4. Driessen et al., Arch. Pharmacol., 341, R104 (1990)
5. The Abstracts of the VIth World Congress on Pain, Apr. 1-6 (1990),
6. Arzneim Forsch. (Drug Res.), 28(I), 183 (1978)),
7. I. Arend et al., Arzneim. Forsch. (Drug Res.), 28(I), 199 (1978),
8. L. Flohe et, al., Arzneim. Forsch. (Drug Res.), 28(I), 213 (1978),
9. T. Yanagita, Arzneim. Forsch. (Drug Res.), 28(I), 158 (1978).
10. J. Jaffe “Goodman and Gilman's, The Pharmacological Basis of Therapeutics”, 8th edition; Gilman et al.; Pergamon Press, New York, 1990; Chapter 22; pages 522-573
11. Woodbury, D. and Fingl, E. in “The Pharmacological Basis of Therapeutics”, 5th Ed.; Goodman, L. and Gilman, A., Chapter 15, (1975)¹¹.
12. Takemori, Annals New York Acad. Sci., 281,262 (1976)
13. R. Taber et al., J. Pharm. Expt. Thera., 169(1), 29 (1969)
14. Pircio et al., Arch. Int. Pharmacodyn., 235, 116 (1978)
15. G. Stacher et. al., Int. J. Clin. Pharmacol. Biopharmacy, 17, 250 (1979)
16. M. J. S. Langman, Am. J. Med. 84 (Suppl. 2A): 15-19, 1988)¹⁶; P. A. Inselin “The Pharmacological Basis of Therapeutics” 8th Ed.; Gilman, A. G. et al., Chapter 26, pp. 664-668, 1990
17. W.O. 0029022A1
18. W.O. 06053012
19. W.O. 09850075
20. E.P. 08459989B1

21. W.O. 05107467

TABLE 4
Patient Characteristics

| | Treatment A | Treatment B | Treatment C | Treatment D | Treatment E |
|---|---|---|---|---|---|
| Characteristic | N = 10 | N = 10 | N = 10 | N = 10 | N = 10 |
| Mean age +/− SD (y) | 44.2 ± 13 | 42.1 ± 11 | 42.5 ± 12 | 41.2 ± 12 | 44.2 ± 11 |
| Sex (M/F) | 23/10 | 20/17 | 22/11 | 19/18 | 16/15 |

Treatment A; Tramadol hydrochloride 100 mg
Treatment B: Neproxen 250 mg
Treatment C: Example 1
Treatment D: Ultram ER 100 mg + Naproxen 250 mg
Treatment E: Example 2

TABLE 5

| | | Pain scored by VAS | | | |
|---|---|---|---|---|---|
| | Treatment A | Treatment B | Treatment C | Treatment D | Treatment E |
| VAS Time hours | N = 10 | N = 10 | N = 10 | N = 10 | N = 10 |
| 0 | 32.4 ± 21.2 | 33.9 ± 19.1 | 33.7 ± 18.2 | 31.9 ± 19.1 | 30.8 ± 21.1 |
| 3 | 23.1 ± 20.1 | 18.5 ± 19.1 | 24.8 ± 19.1 | 25.1 ± 17.8 | 24.2 ± 22.8 |
| 6 | 18.8 ± 20.6 | 13.9 ± 21.1 | 21.1 ± 22.9 | 22.2 ± 18.6 | 23.3 ± 19.3 |
| 9 | 14.9 ± 17.2 | 13.8 ± 20.3 | 14.2 ± 23.1 | 15.3 ± 20.6 | 13.9 ± 19.6 |
| 12 | 13.1 ± 21.2 | 14.6 ± 18.6 | 11.2 ± 20.9 | 11.3 ± 20.2 | 12.3 ± 16.2 |
| 15 | 13.9 ± 19.2 | 17.1 ± 21.5 | 11.0 ± 16.2 | 9.5 ± 18.9 | 10.1 ± 21.8 |
| 18 | 15.4 ± 21.3 | 18.9 ± 19.7 | 9.5 ± 17.8 | 9.1 ± 22.1 | 9.7 ± 22.1 |
| 21 | 14.5 ± 19.2 | 20.1 ± 18.3 | 10.4 ± 16.2 | 9.3 ± 17.3 | 10.2 ± 8.2 |
| 24 | 13.9 ± 19.2 | 24.8 ± 20.9 | 11.6 ± 16.2 | 10.8 ± 17.5 | 9.3 ± 21.2 |

Values are in means ± SD with ranges in parentheses unless indicated otherwise
Treatment A; Tramadol hydrochloride 100 mg
Treatment B: Neproxen 250 mg
Treatment C: Example 1
Treatment D: Ultram ER 100 mg + Naproxen 250 mg
Treatment E Example 2

TABLE 6
Dissolution Study Conditions

| | |
|---|---|
| Apparatus: | USP basket of 10 Mesh |
| Medium of dissolution | 0.1 N Hydrochloride |
| Vessel Volume | 900 ml |
| Temperature | 37′-38″ C. |
| Wavelength | 271 nm |
| Flow Cell Measurement | 1 CM |
| Speed | 75 RPM |
| Run time | 900 minutes |
| Intervel for sampling | 30 Minutes |

1. A slow release pharmaceutical composition comprising
a. a core comprising therapeutically effective amount of Tramadol or its pharmaceutically equivalent salt thereof and at least one pharmaceutically acceptable excipient,
b. A slow release coat,
c. An immediate release layer comprising therapeutically effective amount of Naproxen or its pharmaceutically equivalent salt thereof and at least one pharmaceutically acceptable excipient.

2. A slow release pharmaceutical composition of claim 1 wherein tramadol having an in vitro dissolution rate, when measured using the paddle Method at 100 rpm in 900 ml 0.1 N HCl, 37° C., from about 0% up to about 50% of tramadol is released after 1 hour, from about 0% up to about 75% of tramadol is released after 2 hours, from about 3% up to about 50% of tramadol is released after 4 hours, from about 10% up to about 75% of tramadol is released after 8 hours, from about 25% up to about 100% of tramadol is released after 10 hours, from about 50% up to about 100% of tramadol is released after 12 hours, and from about 65% up to about 100% of tramadol released after 24 hours.

3. A slow release pharmaceutical composition of claim 1 wherein tramadol having an in vitro dissolution rate, when measured using the paddle Method at 100 rpm in 900 ml 0.1 N HCl, 37° C., from about 0% up to about 20% of tramadol is released after 1 hour, from about 2% up to about 20% of tramadol is released after 2 hours, from about 3% up to about 50% of tramadol is released after 4 hours, from about 10% up to about 75% of tramadol is released after 8 hours, from about 25% up to about 100% of tramadol is released after 10 hours, from about 50% up to about 100% of tramadol is released after 12 hours, and from about 65% up to about 100% of tramadol released after 24 hours.

4. A slow release pharmaceutical composition of claim 1 wherein tramadol having an in vitro dissolution rate, when measured using the paddle Method at 100 rpm in 900 ml 0.1 N HCl, 37° C., from about 0% up to about 10% of tramadol is released after 1 hour, from about 2% up to about 10% of tramadol is released after 2 hours, from about 3% up to about 50% of tramadol is released after 4 hours, from about 10% up to about 75% of tramadol is released after 8 hours, from about 25% up to about 100% of tramadol is released after 10 hours, from about 50% up to about 100% of tramadol is released after 12 hours, and from about 65% up to about 100% of tramadol released after 24 hours.

5. A slow release pharmaceutical composition of claim 1 wherein the slow release coat comprises at least one hydrophilic polymer.

6. A slow release pharmaceutical composition of claim 1 wherein the core comprises at least one hydrophobic polymer.

7. A slow release pharmaceutical composition of claim 1 wherein the tramadol is present in an amount from about 25 mg to about 500 mg.

8. A slow release pharmaceutical composition of claim 1 wherein the Neproxen is present in an amount from about 25 mg to about 600 mg.

9. A slow release pharmaceutical composition comprising
a. A core comprising tramadol hydrochloride, polyvinylpyrrolidone or polyvinyl alcohol and microcrystalline cellulose,
b. A slow release coat comprising hydroxypropylmethylcellulose or polyvinylpyrrolidone, and polyethylene glycol
c. An immediate release layer comprising a therapeutically effective amount of a naproxen or its pharmaceutically equivalent salt thereof and at least one pharmaceutically acceptable excipient.

10. A slow release pharmaceutical composition of claim 9 wherein the tramadol is present in an amount from about 25 mg to about 500 mg.

11. A slow release pharmaceutical composition of claim 9 wherein the Neproxen is present in an amount from about 25 mg to about 600 mg.

12. A slow release pharmaceutical composition of claim 9 wherein the composition is a tablet or a capsule or a suppository

13. A slow release pharmaceutical composition comprising
a. a core comprising therapeutically effective amount of Tramadol or its pharmaceutically equivalent salt thereof and a at least one pharmaceutically acceptable excipient,
b. A slow release coat,
c. An immediate release layer comprising therapeutically effective amount of naproxen or its pharmaceutically equivalent salt thereof and at least one pharmaceutically acceptable excipient
wherein the preparation is suitable for dosing either once a day or twice a day.

14. A slow release pharmaceutical composition of claim 13 wherein the tramadol is present in an amount from about 25 mg to about 500 mg.

15. A slow release pharmaceutical composition of claim 13 wherein the Naproxen is present in an amount from about 25 mg to about 600 mg.
